(19) United States
(12) Patent Application Publication
(10) Pub. No.: US 2002/0077504 A1
(43) Pub. Date: Jun. 20, 2002

(54) Catalyst for the hydrogenation of aromatic nitro compounds

(76) Inventors: Peter Albers, Hanau (DE); Emmanuel Auer, Frankfurt (DE); Michael Gross, Frankfurt/Main (DE); Jurgen Krauter, Hosbach (DE); Uwe Packruhn, Frankfurt/Main (DE)

Correspondence Address: SMITH, GAMBRELL & RUSSELL, LLP, ATTORNEYS AT LAW, SUITE 800, 1850 M STREET, N.W., WASHINGTON, DC, 20036, US

(21) Appl. No.: 09/962,801

(22) Filed: Sep. 26, 2001

(30) Foreign Application Priority Data
Sep. 28, 2000 (EP) 00 121 075.6

(51) Int. Cl. C 07C 2 9/36

(52) U.S. Cl. 564/423; 502/326

(57) ABSTRACT

Hydrogenation catalyst containing, as the carbon support, a carbon black with an H content of >4000 ppm and, as the catalytically active component, palladium and/or platinum or bi- or multi-metallically doped or alloyed palladium and/or platinum is prepared by addition of metal salt solutions to a suspension of the carbon black with an H content of >4000 ppm, hydrolyzing the metal salt solutions by using a basic compound and carrying out complete deposition of the metal by reduction with a reducing agent. The hydrogenation catalyst can be employed for the hydrogenation of nitroaromatics.

### INTRODUCTION AND BACKGROUND

The present invention relates to a pulverulent hydrogenation catalyst, a process for its preparation and its use in catalytic suspension hydrogenation of aromatic nitro compounds.

Aromatic amines are currently central units in the preparation of polymers, rubber products, agricultural and pharmaceutical chemicals. Aniline and toluenediamine in particular are important intermediates in the synthesis of iso- and/or diisocyanates, which are used as monomers for the preparation of polyurethanes in the form of various materials (foams, elastomers).

MDA (methylenedianiline), a condensation product of 2 mol aniline with formaldehyde, and bis-para-amino-cyclohexylmethane (PACM), the secondary product stereoselectively hydrogenated on the ring, are moreover widely used in the polymer industry.

A number of different processes and/or catalysts are currently used on an industrial scale for the preparation of aromatic amines by catalytic hydrogenation of the corresponding nitro compound. In addition to gas phase hydrogenation of nitrobenzene, which is employed in particular for the preparation of aniline, there are a number of processes which operate in the liquid phase. Both base metal catalysts on SiO₂ supports and activated metal catalysts of the Raney® type are used here.

The use of catalysts containing noble metals for catalytic hydrogenation of aromatic nitro compounds in the liquid phase has been known for a long time (G. C. Bond, P. B. Walls, Advan. Catal. Relat. Subj. 15, 1964, 92).

Although palladium catalysts are widely used industrially for the catalytic hydrogenation both of nitrobenzene (NB) to aniline and of dinitrotoluene (DNT) to toluenediamine (TDA), one of the central problems is deactivation of the catalyst due to the formation of undesirable by-products, such as, for example, derivatives hydrogenated on the ring or dimerization and/or oligomerization products of partly hydrogenated intermediate products of the reaction, which are summarized in the literature by the term “tar formation”.

A number of publications are known which are concerned with methods for increasing the selectivity in the liquid phase hydrogenation of aromatic nitro compounds and at the same time for improving the yield of amine by choice of a suitable support material and modification of the palladium catalysts with iron or other metals.

The use of modified palladium catalysts is known (EP 002 308 B1). A very high dispersion of the palladium on the support surface is achieved here by the use of an active charcoal support of high surface area. This leads to an improvement in the activity in the catalytic hydrogenation of dinitrotoluene, which is carried out in methanol or other suitable solvents.

The very good dispersion of the noble metal on the support contributes in particular to a hydrogenation reaction which proceeds selectively, since the high heat effect of the nitro group hydrogenation can lead to the formation of undesirable by-products (“over-hydrogenation”). A high dispersion of the noble metal on the support is therefore necessary for immediate dissipation of the exothermicity at the reaction center occurring during the hydrogenation to the support material (removal of heat).

It is furthermore known to prepare an iron-modified palladium catalyst on a hydrophobic carbon black support (“oleophilic carbon black”) (U.S. Pat. No. 3,127,356). In particular, the use of a very finely divided carbon black support, such as acetylene black, and the doping of the palladium with elements such as iron and/or platinum in the known process leads to a significant improvement in the activity rate (conversion of dinitrotoluene with respect to the metal employed) and to an increase in selectivity.

The choice of a suitable carbon black support has the advantage that due to the high thermal conductivity of the support material, compared with active charcoal, rapid removal of the exothermicity is possible.

Nevertheless, the oleophilic acetylene black described in U.S. Pat. No. 3,127,356 has the disadvantage that a highly dispersed deposition of the metals palladium, platinum and iron in aqueous suspension is not possible in an optimum manner because of the hydrophobic surface of the carbon black. For this reason, catalysts which are prepared on an acetylene black (for example Shawinigan Black from Chevron) according to example 1 of U.S. Pat. No. 3,127,356 have only a limited activity and selectivity in the catalytic hydrogenation of dinitrotoluene.

An object therefore of the present invention is to prepare a hydrogenation catalyst on a carbon black support which has a higher dispersion of the noble metal and is more active and selective than the known catalysts.

### SUMMARY OF THE INVENTION

The above and other objects can be achieved according to the present invention by a hydrogenation catalyst comprising, as the carbon support, a carbon black with an H content of >4000 ppm, preferably >4200 ppm, particularly preferably >4400 ppm, determined by CHN analysis, and, as the catalytically active component, palladium and/or platinum or bi- or multi-metallically doped or alloyed palladium and/or platinum.

Bi- and/or multi-metallically doped or alloyed palladium and/or platinum can be obtained by doping the palladium and/or platinum or alloys of palladium and/or platinum with the elements Fe, V, Rh, Sn, Ru or combinations thereof.

The ratio of CTAB surface area (cetylammonium bromide) to BET surface area can be 0.9-1.1 in the carbon black according to the invention.

A CTAB/BET surface area ratio of the carbon black of close to 1 allows highly dispersed deposition of active metal components on the support without noble metal crystallites being deposited in the pores of the carbon black support and its specific metal surface no longer being accessible to substrate molecules because of mass transfer limitation.

A carbon black with an H content of greater than 4000 ppm and a peak integral ratio, determined by inelastic neutron scattering (INS), of non-conjugated H atoms (1250-2000 cm⁻¹) to aromatic and graphitic H atoms (1000-1250 cm⁻¹ and 750-1000 cm⁻¹) of less than 1.22, preferably less than 1.20, can preferably be employed as the carbon black with an H content of greater than 4000 ppm, determined by CHN analysis.

The preparation of the furnace black can be carried out in a carbon black reactor which comprises a combustion zone, a reaction zone and a termination zone along the reactor axis. In the combustion zone, a flow of hot waste gas is generated by complete combustion of a fuel in an oxygen-containing gas. Carbon black raw materials are then mixed into the hot waste gas in the reaction zone. The formation of carbon black is stopped in the termination zone by spraying in water, a liquid and gaseous carbon black raw material being sprayed in at the same point.

The liquid carbon black raw material can be atomized by pressure, steam, compressed air or the gaseous carbon black raw material.

Liquid hydrocarbons burn more slowly than gaseous ones, since they must first be converted into the gas form, that is to say vaporized. As a result, the carbon black has contents formed from the gas and those formed from the liquid.

The so-called K factor is often used as a standard value for characterizing the excess air. The K factor is the ratio of the amount of air required for stoichiometric combustion of the fuel to the amount of air actually fed to the combustion. A K factor of 1 therefore means a stoichiometric combustion. hi the case of an excess of air, the K factor is less than 1. As in the case of known carbon blacks, K factors of between 0.3 and 0.9 can be used here. K factors of between 0.6 and 0.7 are preferably used.

Liquid aliphatic or aromatic, saturated or unsaturated hydrocarbons or mixtures thereof, distillates from coal tar or residual oils which are formed during catalytic cracking of petroleum fractions or in olefin production by cracking of naphtha or gas oil can be employed as the liquid carbon black raw material.

Gaseous aliphatic, saturated or unsaturated hydrocarbons, mixtures thereof or natural gas can be employed as the gaseous carbon black raw material.

### BRIEF DESCRIPTION OF DRAWINGS

The present invention will be further understood with reference to the drawings, wherein

FIG. 1 is a schematic diagram of a carbon black reactor used to prepare the carbon black of the invention;

FIG. 2 is a schematic sectional view of the lance contained in the combustion chamber;

FIG. 3 is a spectra of inelastic neutron scattering for a commercial carbon black and the carbon black of the present invention; and

FIG. 4 is the INS spectra of two platinum catalysts on a commercial carbon black and the carbon black of the invention.

### DETAILED DESCRIPTION OF THE INVENTION

The process described is not limited to a particular reactor geometry. Rather, it can be adapted to various reactor types and reactor sizes. Both pure pressurized atomizers (one-component atomizers) and two-component atomizers with internal or external mixing can be employed as the carbon black atomizer, it being possible for the gaseous carbon black raw material to be used as the atomizing medium. The combination described above of a liquid with a gaseous carbon black raw material can thus be realized, for example, by using the gaseous carbon black raw material as the atomizing medium for the liquid carbon black raw material.

Two-component atomizers can preferably be employed for atomizing liquid carbon black raw material. While in one-component atomizers a change in throughput also leads to a change in droplet size, the droplet size in two-component atomizers can be influenced largely independently of the throughput.

The CTAB surface area can be from 20 to 400 m²/g, preferably 20 to 150 m²/g. The DBP number can be from 40 to 200 ml/100 g, preferably 100 to 180 ml/100 g.

A carbon black known from DE 19521565 can furthermore be employed as the carbon black with a hydrogen content of >4000 ppm, determined by CHN analysis.

The carbon blacks can be employed in untreated or after-treated form. The carbon black can be non-doped or doped with foreign atoms. Foreign atoms can be Si, Zr, Sb, V, Fe, Mg or Ti.

The very high hydrogen content is an indication of a severe disturbance in the carbon lattice due to an increased number of edges of the C crystallites, which are smaller compared with acetylene black (for example Shawinigan Black). The hydrogen content can be determined beyond doubt by neutron diffraction and indicates the existence of sp³-hybridized C atoms, so-called defects in the crystallite lattice, on which palladium, iron or platinum can be preferentially deposited.

For optimum functioning of the hydrogenation catalysts according to the invention, the loading can be between 0.05 and 80 wt. % palladium and/or platinum, preferably between 0.5 and 10 wt. %, based on the total weight of the catalyst.

The atomic ratios between palladium and/or platinum and the other doping and/or alloying elements, of which there are optionally several, can be between 200:1 and 1:200, but preferably between 100:1 and 1:100.

In the case of tri- or multi-metallic hydrogenation catalysts, the atomic ratio of the further alloying components with respect to one another can be varied within the limits of between 100:0 and 0:100. However, atomic ratios within the limits of 50:1 and 1:50 are particularly advantageous.

The invention also provides a process for the preparation of the hydrogenation catalyst according to the invention, which is characterized in that noble metal salt solution and optionally salt solutions of the doping or alloying elements are added simultaneously, in succession or in a two-stage process after prior preparation of a noble metal pre-catalyst to a suspension of a carbon black with an H content of >4000 ppm, the (noble) metal salt solutions are precipitated in hydrolyzed form as hydroxides on the support using a basic compound, and complete deposition of the noble metal and the other metals is carried out by reduction with a reducing agent. The reduction can be carried out at a temperature of 0 to 100° C.

The reduction with hydrogen gas can optionally be carried out in the liquid phase or on the dried catalyst. The sequence in which the support material, water, metal salt solutions and water-soluble reducing agents are brought together can also be varied. Formaldehyde, hydrazine or sodium borohydride, for example, can be used as suitable wet chemistry reducing agents.

The use of a reducing agent is optional, i.e. the catalyst according to the invention can also be separated off from the reaction mixture by filtration after the hydrolysis of the (noble) metal salt solutions without further reduction.

After the catalyst has been separated off by filtration, a drying step can follow. After the preparation by a wet chemistry method, a heat treatment under an inert gas or a reducing atmosphere at temperatures of between 0° C. and 1000° C., preferably between 100° C. and 700° C., can furthermore be carried out.

The hydrogenation catalyst according to the invention can be employed for the hydrogenation of nitroaromatics. The catalyst according to the invention can be employed in particular for the hydrogenation of nitrobenzene to aniline and of dinitrotoluene to toluenediamine.

The catalytic hydrogenation of the nitro compounds can be carried out in the liquid phase as a continuously or discontinuously operated process under pressures of between 1 and 100 bar at temperatures of between 0° C. and 250° C. in the presence of the catalyst according to the invention.

The catalytic hydrogenation of nitrobenzene or dinitrotoluene in the presence of the catalyst according to the invention can be carried out in a discontinuous or continuously operated stirred reactor in the presence of a solvent, such as, for example, methanol or toluene. It can also be carried out in a mixture of aniline/water or toluenediamine/water, especially in the case of continuous processes.

The hydrogenation of dinitrotoluene to toluenediamine can be carried out at temperatures of between 70 and 200° C., preferably 90 and 150°, under pressures of between 1 and 100 bar, preferably 25 to 50 bar. If the hydrogenation is operated continuously, the amount of deduct reacted must be replaced by topping up and the product/water mixture must be removed from the reactor.

The hydrogenation according to the invention to toluenediamnine in the presence of the catalyst according to the invention is advantageously distinguished above all by a low formation of by-products and high yields of toluenediamine. The undesirable formation of derivatives hydrogenated on the ring and incompletely hydrogenated intermediates is not observed. The di- and oligomerization of various intermediate stages of the reaction (“tar formation”) is also significantly lower. The yield of toluenediamine is in all cases above those which it has been possible to achieve using known palladium or modified palladium catalysts on carbon black supports.

The catalysts according to the invention are distinguished by a high dispersion of the metal particles deposited on the support and a higher activity and selectivity in the catalytic hydrogenation of nitroaromatic compounds (for example aniline, dinitrotoluene).

### EXAMPLES

In the following examples and comparison examples, hydrogenation catalysts according to the invention and comparison catalysts are prepared and are compared with one another in respect of their catalytic properties in the hydrogenation of nitroaromatics.

The carbon black B2 from Degussa-Hüls is employed as the support material for the catalyst according to the invention, and the acetylene black Shawinigan Black from Chevron for the comparison catalysts.

Preparation of the carbon black:

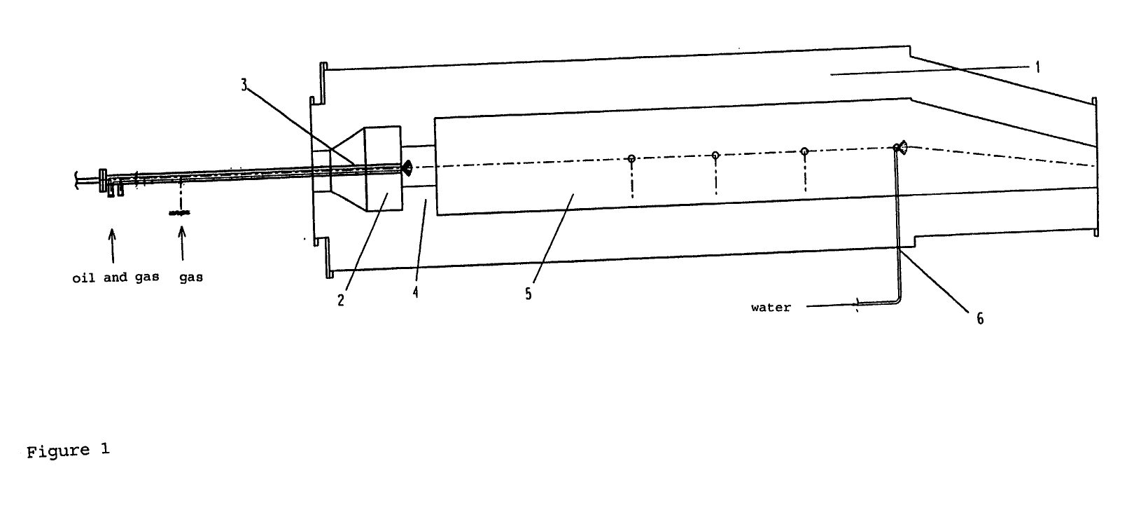

The carbon black B2 is prepared in the carbon black reactor 1 shown in FIG. 1 by spraying the liquid and gaseous carbon black raw material in at the same point. This carbon black reactor 1 has a combustion chamber 2. The oil and gas are introduced into the combustion chamber via the axial lance 3. The lance can be displaced in the axial direction to optimize the carbon black formation.

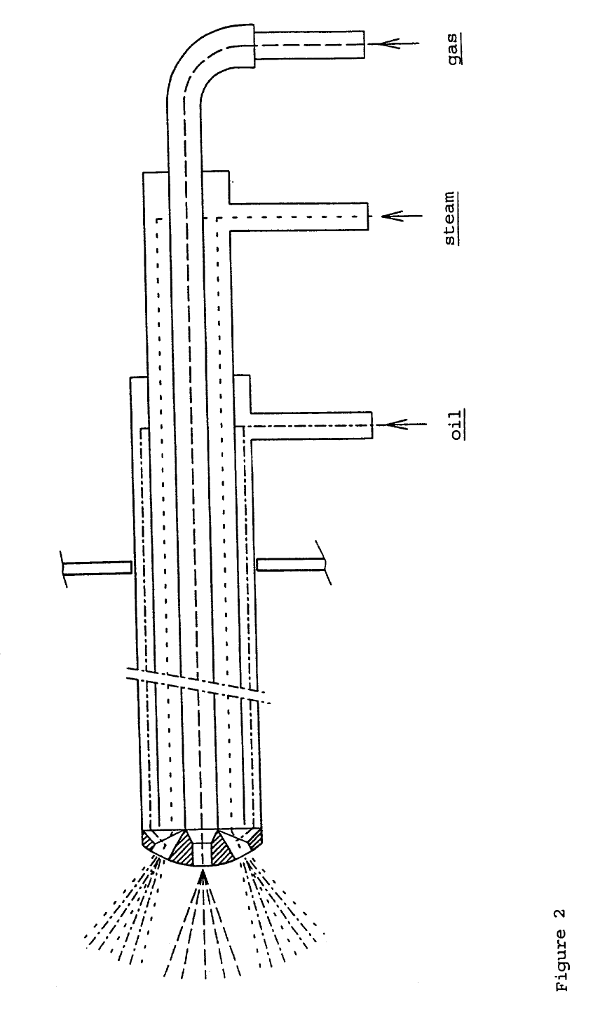

The combustion chamber runs to the narrow zone 4. By passing through the narrow zone, the reaction gas mixture expands into the reaction chamber 5. The lance has suitable spray cans on its head (FIG. 2).

The combustion zone, reaction zone and termination zone which are important for the process cannot be separated sharply from one another. Their axial extension depends on the particular positioning of the lances and the quenching water lance 6.

The dimensions of the reactor used can be seen from the following list:

| | |
|---|---|
| Largest diameter of the combustion chamber: | 696 mm |
| Length of the combustion chamber to the | 630 mm |
| narrow zone: | |
| Diameter of the narrow zone: | 140 mm |
| Diameter of the reaction chamber: | 802 mm |
| Position of the oil lances | + 160 mm |
| Position of the quenching water lances¹⁾ | 2060 mm |

¹⁾measured from the zero point (start of the narrow zone)

The reactor parameters for the preparation of the carbon black according to the invention are listed in the following table.

| | |
|---|---|
| Reactor parameters | Carbon black |

| Parameter | Unit | B2 |
|---|---|---|
| Combustion air | Nm³/h | 1500 |
| Termperature of | ° C. | 550 |
| the combustion | | |
| Σ natural gas | Nm³/h | 156 |
| K factor (total) | | .070 |
| Carbon black oil, | kg/h | 670 |
| axial | | |
| Carbon black oil | Mm | +16 |
| position | | |
| Atomizer vapour | kg/h | 100 |
| Additive (K₂CO₃ | 1/h × g/l | 5.5 × 3.0 |
| solution) | | |
| Additive position | | axial |
| Reaction exit | ° C. | 749 |
| Quenching position | mm | 9/8810 |

Characterization of the support materials:

The hydrogen contents of the two carbon blacks are determined both by CHN elemental analysis and by means of neutron diffraction. The method of inelastic neutron scattering (INS) is described in the literature (P. Albers, G. Prescher, K. Seibold, D. K. Ross and F. Fillaux, Inelastic Neutron Scattering Study Of Proton Dynamics In Carbon Blacks, Carbon 34 (1996) 903 and P. Albers, K. Seibold, G. Prescher, B. Freund, S. F. Parker, J. Tomkinson, D. K. Ross, F. Fillaux, Neutron Spectroscopic Investigations On Different Grades Of Modified Furnace Blacks And Gas Blacks, (Carbon 34 (1999) 437).

The INS (or IINS—inelastic, incoherent neutron scattering) method offers some quite unique advantages for still more intensive characterization of carbon blacks and active charcoals.

As an addition to the proven quantification of the H content by elemental analysis, the INS method enables the sometimes quite low hydrogen content in graphitized carbon blacks (approx. 100-205 ppm), carbon blacks (approx. 2000-4000 ppm in furnace blacks) and in active charcoals (approx. 5000-12000 ppm in typical catalyst supports) to be broken down into a more detailed form in respect of its bonding states.

For comparison purposes, the values of the total hydrogen content of the carbon blacks determined by means of CHN analysis (Leco-404 analyzer with a thermal conductivity detector) are listed in the following table. The spectra integrals standardized to the sample weight are also stated, these being determined as follows: Integration of the range of an INS spectrum of 500-3600 cm⁻¹. As a result of this, the graphite vibration band of the carbon matrix at approx. 110 cm⁻¹ is cut out.

| | H content | H content |
|---|---|---|
| | [ppm] by CHN elemental | [integral/sample weight] |
| Carbon black | analysis | by INS |
| B2 | 4580 ± 300 | 69.1 |
| Shawinigan Black | 800 | 46.5 |
| acetylene black | | |

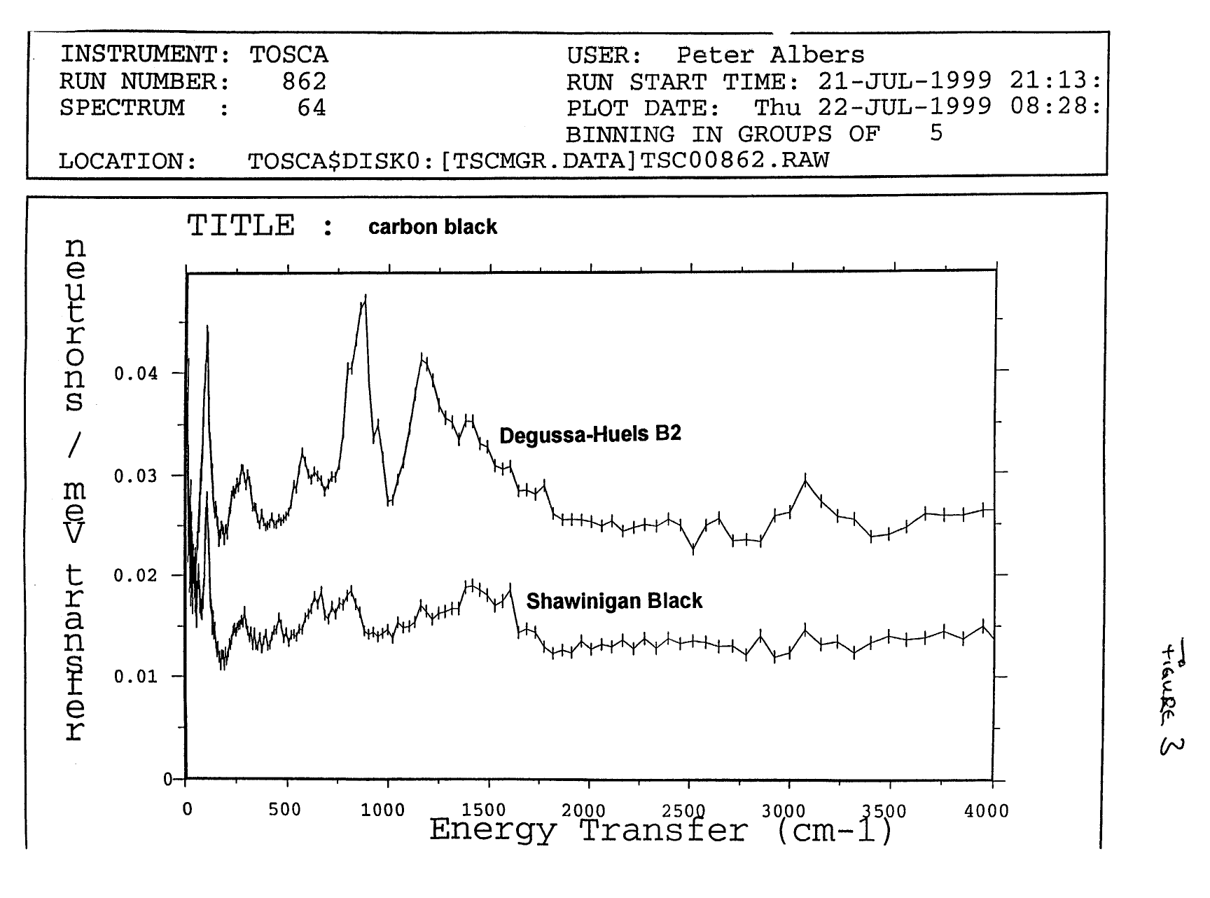

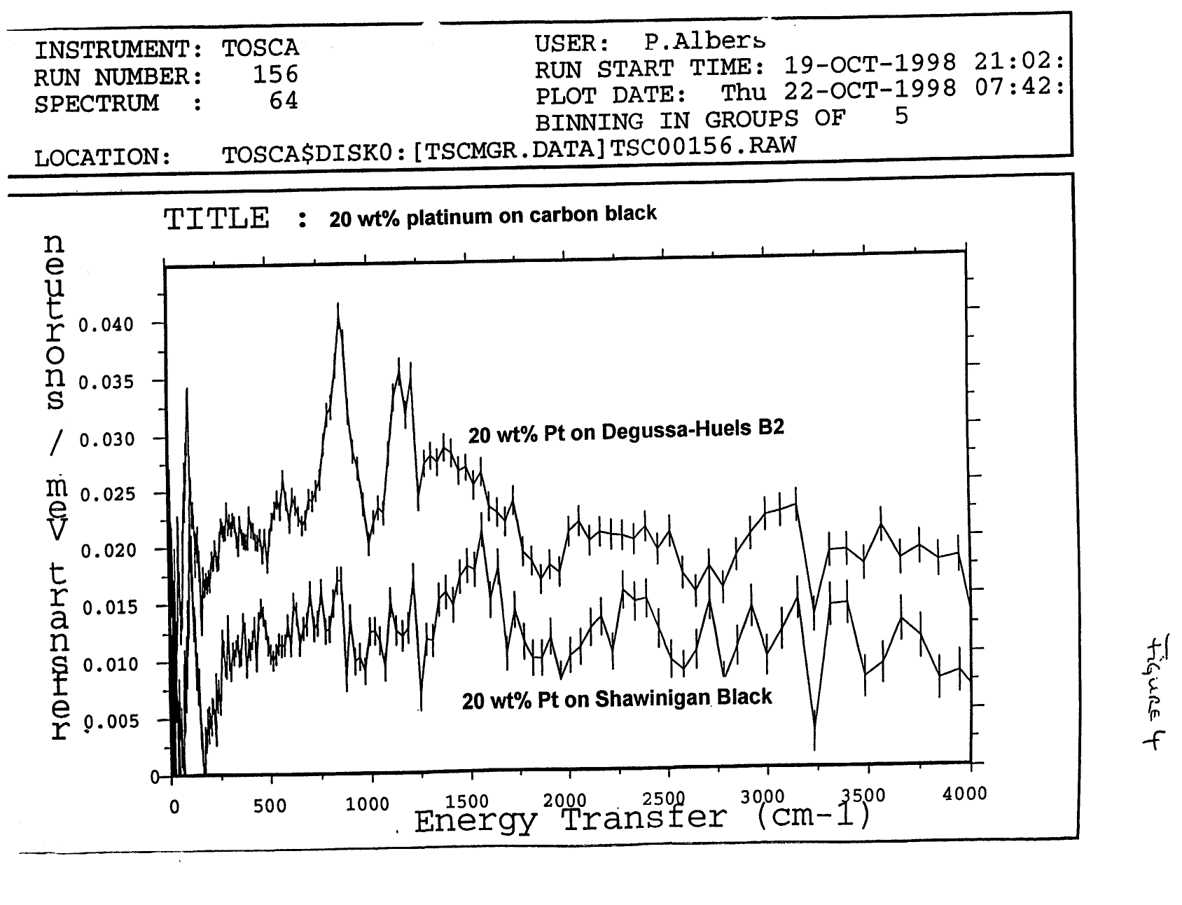

The spectra of the inelastic neutron scattering for Shawinigan Black and carbon black B2 are shown by way of example in FIG. 3. FIG. 4 shows the INS spectra of two platinum catalysts on Shawinigan Black and the Degussa-Hüls carbon black B2 (catalyst according to the invention) respectively.

The differences in the vibration range for the Cₛₚ₃-H vibration of the two carbon blacks can be clearly seen.

For comparison of the materials of the two carbon blacks, the following ranges are important—in addition to the graphite vibration band at 112 cm⁻¹:

1. the range of 750-1000 cm⁻¹ (i.e. up to the sharp separation at 1000 cm⁻¹); it corresponds to the “out of plane” C-H deformation vibration bands at the truncation edges of the lattice planes of the graphitic carbon black units.

2. the range of 1000-1250 cm⁻¹; this corresponds to the “in plane” C-H deformation vibration bands

3. the range of 1250-2000 cm⁻¹; this corresponds to the C-H deformation vibrations of non-conjugated constituents.

In addition to the range integrals of the spectra segments mentioned, some quotients of these values are also given tentatively in the following table; comparison with the total hydrogen content furthermore shows a quite satisfactory correlation between these INS values and the results of the CHN analysis:

| | | | | | | H |
|---|---|---|---|---|---|---|
| | A | B | C | C/A | C/(A + B) | content |
| | 750- | 1000- | 1250- | | | Ppm |
| | 1000 | 1250 | 2000 | | | |
| | cm⁻¹ | cm⁻¹ | cm⁻¹ | | | |
| | ±1 | ±1 | ±3 | | | |
| Shawinigan | 24 | 24 | 76 | 3.2 | 1.58 | 800 |
| Degussa-Hüls | 107 | 99 | 241 | 2.25 | 1.17 | 4580 |
| B2 | | | | | | |

Results of the spectra integrations: Comparison of the range integrals with the total hydrogen content according to CHN analysis.

Due to the significantly higher hydrogen content, the carbon black B2 according to the invention is significantly more hydrophilic than Shawinigan Black, which promotes highly dispersed deposition of the noble metal. Other properties of the two carbon blacks, e.g. the surface ratio of the specific total surface area (determined by BET) and the CTAB surface area (determined by cetylammonium bromide adsorption in accordance with DIN 66132) are very similar.

| | CTAB | BET | BET:CTAB |
|---|---|---|---|
| | surface area | surface area | surface area |
| Carbon black | [m²/g] | [m²/g] | ratio |
| B2 | 40 | 40 | 1 |
| Shawinigan | | 80 | 1 |
| Black | | | |

### Examples:

1. Palladium on Shawinigan Black (comparison example) 23.6 g Shawinigan Black carbon black are suspended in deionized water and the pH is rendered alkaline (pH=10.0) with sodium carbonate solution. 0.44 g palladium(II) chloride solution (20%) is added to this suspension. After heating up to 90° C., a pH of 6.5 is established. The mixture is subsequently stirred and the catalyst is filtered off. The finished catalyst comprises 1.75 wt. % palladium.

2. Palladium on carbon black B2 according to the invention A palladium catalyst is prepared analogously to example 1 using the Degussa-Hüls carbon black B2. The metal loading on the support is also 1.75 wt. %.

3. Pd catalyst on Shawinigan Black according to U.S. Pat. No. 3,127,356 A mono-metallic Pd catalyst on Shawinigan Black is prepared according to example 1 of U.S. Pat. No. 3,127,356. As a modification of the preparation instructions, only palladium(II) chloride, but not an iron compound, is used for the preparation.

4. Palladium/iron on Shawinigan Black 23.6 g Shawinigan Black carbon black are suspended in deionized water and the pH is rendered alkaline (pH=10.0) with sodium carbonate. 1.05 g iron(III) chloride in 100 ml deionized water and 0.44 g palladium(II) chloride (20%) are added to this suspension. After heating up to 90° C., a pH of 6.5 is established. The mixture is subsequently stirred and the catalyst is filtered off. The finished catalyst comprises 1.75 wt. % palladium and 4.2 wt. % iron.

5. Pd/Fe on Degussa-Hüls carbon black B2 (according to the invention) A Pd/Fe catalyst with a loading of 1.75 wt. % Pd and 4.2 wt. % Fe is prepared analogously to example 4 using Degussa-Hüls carbon black B2.

6. Pd/Fe catalyst on Shawinigan Black according to U.S. Pat. No. 3,127,356 A bimetallic Pd/Fe catalyst on Shawinigan Black is prepared according to example 3 of U.S. Pat. No. 3,127,356.

7. Pt on Shawinigan Black 24.75 g Shawinigan Black carbon black are suspended in deionized water and the pH is rendered alkaline (pH=8.0) with sodium bicarbonate. After heating up to 90° C., 1.00 g hexachloroplatinic(IV) acid (25%) is added to this suspension. The pH is rendered alkaline again (pH>8.0) and reduction is carried out at 90° C. with formaldehyde solution (37%). The mixture is subsequently stirred and the catalyst is filtered off. The finished catalyst comprises 1 wt. % platinum.

8. Pt on Degussa-Hüls carbon black B2 (according to the invention) A platinum catalyst with a noble metal loading of 1 wt. % is prepared analogously to example 7 using Degussa-Hüls carbon black B2.

Characterization of the catalysts

The hydrogenation catalysts prepared were characterized in respect of their noble metal dispersion via CO chemisorption and of their activity in the nitrobenzene low pressure test.

| | CO chemi- | | Nitrobenzene |
|---|---|---|---|
| Catalyst/ | sorption | Dispersion | activity [mg/g |
| noble metal content | [ml/g] | [%] | min] |
| Example 1 | 0.98 | 26.4 | 920 |
| 1.75% Pd | | | |
| Example 2 | 1.05 | 29.0 | 1000 |
| 1.75% Pd | | | |
| Example 3 | 0.49 | 13.2 | 650 |
| 1.75% Pd | | | |
| Example 4 | n.d.* | n.d.* | 60 |
| 1.75% Pd 4.2% Fe | | | |
| Example 5 | n.d.* | n.d.* | 80 |
| 1.75% Pd 4.2% Fe | | | |
| Example 6 | n.d.* | n.d.* | 20 |
| 1.75% Pd 4.2% Fe | | | |
| Example 7 | 0.54 | 47.0 | 300 |
| 1% Pt | | | |
| Example 8 | 0.87 | 76.0 | 650 |
| 1% Pt | | | |

n.d.* in the case of bimetalllic Pd-Fe catalysts highly toxic iron carbonyls which falsify the measurement may form during the CO chemisorption measurement. The dispersion of the metal is therefore not stated for these catalysts, since the data are only of limited conclusiveness.

The following reaction parameters apply in the nitrobenzene low pressure test:

| | |
|---|---|
| Reaction pressure | 10 mbar |
| Temperature | 30° C. |
| Solvent | isopropanol/water = 4:1 |
| Stirrer speed | 2000 rpm |

In each case 10 ml nitrobenzene are introduced with 200 mg catalyst in 150 ml solvent into a glass apparatus flushed with hydrogen. The reaction and therefore the uptake of hydrogen is started by switching on the stirrer. After a pre-running time of 3 minutes, the activity in the unit [ml H₂/min·g] is measured for 5 minutes by means of a mass flow meter.

Use examples

The catalysts according to examples 1 to 8 are tested in respect of their activity and selectivity in the discontinuously conducted hydrogenation of dinitrotoluene to toluenediamine. The following reaction parameters are adhered to here:

| | |
|---|---|
| Reaction pressure | 10 bar |
| Temperature | 100° C./120° C. |
| Solvent | toluenediamine/water |

In each case 40 g dinitrotoluene, dissolved in 160 g toluenediamine/>36% water, are hydrogenated quantitatively. The end point of the reaction can be determined precisely by the rapid drop in the uptake of hydrogen to zero. An amount of catalyst of 0.5 wt. %, based on the dinitrotoluene employed, is always used. The by-products in the reaction product are then determined by GC (gas chromatography).

The by-products formed are divided into three groups:

By-product 1: Secondary products of dinitrotoluene, methylnitroaniline and toluenediamine hydrogenated on the ring

By-product 2: Incompletely hydrogenated compounds (for example methylnitroaniline)

By-product 3: Dimers, oligomers (“tar formation”)

The samples are taken from the autoclave after a stirring time of 15 minutes after the end of the uptake of hydrogen.

The results of the hydrogenation under 10 bar are summarized in the following table.

| Catalyst | | Yield of | By-product | | |
|---|---|---|---|---|---|
| according to | Hydrogenation | TDA | 1 | 2 | 3 |
| example | time [min] | [%] | [wt. %] | [wt. %] | [wt. %] |
| 1 | 27 | 97.8 | 0.8 | / | 1.4 |
| 2 | 30 | 98.1 | 0.8 | 0.8 | 0.3 |
| 3 | 35 | 92.5 | 2.2 | 1.9 | 3.4 |
| 4 | 26 | 94.4 | / | / | 5.6 |
| 5 | 25 | 98.8 | / | / | 1.2 |
| 6 | 40 | 83.2 | 4.2 | 1.2 | 11.4 |
| 7 | 35 | 98.8 | / | 0.7 | 0.6 |
| 8 | 32 | 99.6 | / | 0.1 | 0.3 |

Further variations and modifications of the foregoing will be apparent to those skilled in the art and are intended to be encompassed by the claims appended hereto.

German priority application 00 121 075.6 is relied on and incorporated herein by reference.

We claim:

1. A hydrogenation catalyst comprising, as the carbon support, a carbon black with an H content of >4000 ppm and, as the catalytically active component, palladium and/or platinum or bi- or multi-metallically doped or alloyed palladium and/or platinum.

2. The hydrogenation catalyst according to claim 1, wherein the palladium and/or platinum is doped or alloyed with an element selected from the group consisting of Ru, Rh, Fe, V, Sn and combinations thereof.

3. The hydrogenation catalyst according to claim 2, wherein the atomic ratio between palladium and/or platinum and optionally the doping or alloying components is between 200:1 and 1:200.

4. The hydrogenation catalyst according to claim 1, wherein palladium and/or platinum is present in an amount of from 0.05 to 80 wt. %, based on the total weight of the catalyst.

5. A hydrogenation catalyst comprising a carbon black support with an H content of >4000 ppm determined by CHN analysis, palladium and/or platinum or bi- or multi-metallically doped or alloyed palladium and/or platinum.

6. The hydrogenation catalyst according to claim 5, wherein the carbon black has an H content of >4200 ppm.

7. The hydrogenation catalyst according to claim 5, wherein the carbon black has an H content of >4400 ppm.

8. The hydrogenation catalyst according to claim 5, wherein the carbon black has a CTAB/BET ratio of 0.9 to 1.1.

9. A process for the preparation of a hydrogenation catalyst according to claim 1, comprising adding a noble metal salt solution and optionally salt solutions of doping or alloying elements simultaneously, in succession or in a two-stage process after prior preparation of a noble metal pre-catalyst to a suspension of a carbon black with an H content of >4000 ppm, hydrolyzing the noble metal salt solution using a basic compound and completely depositing the noble metal and any element by reducing with a reducing agent.

10. The process for the preparation of a hydrogenation catalyst according to claim 9, further comprising after preparation of the hydrogenation catalyst by a wet chemistry method, heating under an inert gas or reducing atmosphere at temperatures of from 0° C. to 1000° C.

11. A process for the preparation of aniline and toluenediamine, comprising catalytically hydrogenating the corresponding nitro compound in the liquid phase as a continuously or discontinuously operated process under pressures of between 1 and 100 bar at temperatures of between 0° C. and 250° C. in the presence of the catalyst according to claim 1.

12. A process for the hydrogenation of a nitroaromatic compound, comprising placing the nitroaromatic compound in the liquid phase and introducing hydrogen as a continuously or discontinuously operated process under pressures of between 1 and 100 bar at temperatures of from 0° C. to 250° C. in the presence of the catalyst according to claim 1.

13. A carbon black with an H content >4000 ppm as determined by CHN analysis.

14. The carbon black of claim 13 wherein the H content is >4200 ppm as determined by CHN analysis.

15. The carbon black of claim 13 wherein the H content is >4400 ppm as determined by CHN analysis.

16. The carbon black of claim 13 wherein the ratio of CTAB surface area to BET surface area is 0.9-1.1.

17. The carbon black of claim 13 which has a peak integral ratio as determined by inelastic neutron scattering of non-conjugated H atoms, 1250-2000 cm⁻¹ to aromatic and graphitic H atoms, 1000-1250 cm⁻¹ and 750-1000 cm⁻¹ of less than 1.22.

18. The carbon black of claim 13 wherein the peak integral ration is less than 1.2.

19. The carbon black according to claim 13 which is finely divided and hydrophilic.

20. A process for preparing a furnace black in a carbon black reactor which has a combustion zone, a reaction zone and a termination zone comprising:
introducing a fuel mixed with an oxygen containing gas into the combustion zone to obtain combustion of said fuel to form a flow of hot waste gas,
introducing carbon black raw materials in atomized form into the hot waste gas, spraying water, into the termination zone to stop formation of carbon black.
